(19) United States
(12) Patent Application Publication
(10) Pub. No.: US 2025/0268641 A1
(43) Pub. Date: Aug. 28, 2025

(54) PLASMA PROBE

(71) Applicant: Erbe Elektromedizin GmbH, Tuebingen (DE)

(72) Inventor: Uwe SCHNITZLER, Tuebingen (DE)

(21) Appl. No.: 19/058,135

(22) Filed: Feb. 20, 2025

(30) Foreign Application Priority Data
Feb. 23, 2024 (EP) 24159492.8

(51) Int. Cl. A61B 18/04; A61B 18/00

(52) U.S. Cl. CPC A61B 18/04; A61B 2018/00077; A61B 2018/00083; A61B 2018/00184

(57) ABSTRACT

An instrument for argon plasma surgery includes a plastic hose and an electrical conductor extending therethrough, which is formed by a thread of carbon fibers or a flexible or rigid body including carbon fibers. The carbon fibers serve for current conduction and thermal cooling of the plasma foot point which forms directly at the end of the electrical conductor at the carbon fibers or in a compact body in contact with the latter. The electrical conductor is arranged inside the lumen of the hose without being centered and thus without forced contact to the inner wall of hose. The high thermal conductivity of the conductor in the longitudinal direction results in a low thermal stress of hose. Due to this construction, the instrument can be provided with halogen-free plastics, advantageous for thermal disposal of the instrument.

### CROSS REFERENCE TO RELATED APPLICATION

This application claims the benefit of European Patent Application No. 24159492.8, filed Feb. 23, 2024, which is incorporated herein by reference in its entirety.

### TECHNICAL FIELD

The invention refers to an instrument for carrying out surgical operations on a human or animal patient. Particularly, the instrument according to the present invention serves for argon plasma coagulation of living tissue.

### BACKGROUND

An instrument for tissue coagulation configured as argon plasma probe is known from EP 3 769 707 A1. This probe comprises a flexible plastic hose through the entire length of which a lumen extends. A supplying apparatus is connected to the proximal end of the hose, via which the hose can be supplied with argon that flows through it in direction toward the distal end. Inside the hose an electrical conductor is arranged, which is connected at the proximal end of the instrument as well to a pole of a generator comprised in the apparatus and can in this manner be supplied with electrical high frequency voltage for plasma generation. The other pole of the generator is connected to a neutral electrode. The electrical conductor extends through the lumen of the hose up to an electrode, which is formed by means of a metal platelet that is supported with both diametrically opposed edges inside the hose and is in this manner centrally supported. A tip of the electrode facing in distal direction serves to supply an electrical discharge and in this manner to ionize the passing argon flow for producing a plasma stream.

For reducing the temperature stress of the hose due to improved cooling of the electrode, the electrode is provided with a coating that shall support the heat distribution and transfer to the passing gas stream. However, the electrode forms a remarkable heat stress for the plastic hose, which requires the use of a thermally robust plastic material for the hose. Such plastic materials often contain chlorine or fluorine compounds that cause difficulties when disposing such instruments. Particularly during combustion of such materials, substances can be created that can be a remarkable environmental burden and a health hazard due to their toxicity and/or durability.

A cold plasma instrument known from US 2021/0145499 A1 has an exit nozzle through which the plasma shall be electrically pulled outwards. Aluminum or also graphene are considered as material for the electrode.

In addition, from the general industrial field, it is known to form compact electrodes from graphite. For this, reference is made to EP 0 476 572 A2 and JP 2018-098122 A.

### SUMMARY

Starting therefrom, it is one object of the invention to provide an instrument with low environmental burden.

This object is solved by means of an instrument as described herein.

The instrument according to the invention is configured for medical treatment of biological tissue. Preferably, it is configured as flexible probe, which can be inserted through the working channel of an endoscope into the body of a patient. However, the instrument can also comprise a non-flexible rigid shank and can be provided with a handle at the proximal end in order to be used as laparoscopic instrument, for example. If desired, it can also be configured with a handle having a short shank for the open surgical use.

The instrument comprises a hose, which is preferably configured in flexible manner in case of the endoscopic use. If the instrument is configured as laparoscopic instrument, the hose can also be configured as rigid tube shank. This also applies if the instrument is configured for the open surgical use.

A lumen extends through the hose, wherein the lumen extends from the proximal end to the distal end of the hose. At its proximal end, connection means are provided in order to be able to connect the hose to a gas supply source and to supply its lumen with a gas stream. In addition, an electrical connection is provided at the proximal end that is configured for supplying an electrical current in an electrical conductor extending through the lumen up to the distal end of the hose. A combined connector can serve as connection means and as electrical connector, which comprises a gas connection pin as well as at least one electrical pin. However, the electrical connection can also be configured separately from the gas connection means as individual connector.

The conductor ending in or at the exit openings consists entirely or partly of carbon fibers or comprises such carbon fibers. The carbon fibers comprise a high thermal conductivity and are therefore suitable to dissipate heat, which is introduced at the end of the conductor due to the plasma that builds up there, in proximal direction away from the distal conductor end and distribute it over a remarkable section of its length, which has an amount of the multiple of the diameter of the conductor. The gas that passes the conductor in opposite direction then efficiently contributes to cooling. A temperature of the conductor results therefrom that is so low that it can be tolerated by not remarkably temperature resistant plastics. As a result, the hose can consist of a low temperature resistant plastic, particularly a halogen-free plastic. The plastic can be particularly free chlorine- and/or fluorine-free. Thus, a simple environmental-friendly disposal of a used instrument by means of combustion is possible. During combustion, no hazardous substances are produced then that are enduring for a long term and toxic or in other manner detrimental to the environment.

This also applies in relation to the electrical conductor consisting of carbon fibers or comprising the latter at least in a relevant amount, which can also be oxidized to CO₂ without producing problematic chemicals, metals or metal oxides thereby. If desired, the instrument according to the invention can be free of nickel, chromium, molybdenum, cobalt, silver, copper or other expensive or toxic metals, particularly heavy metals.

If the carbon fibers are predominantly orientated in direction of the lumen, it caters to the thermal conductivity of the conductor. The thermal conductivity of the conductor thus is carried out substantially in fiber-longitudinal direction, that is in direction of the preferred thermal conductivity of the carbon fiber.

The carbon fibers can be embedded in a body, which is rigid. However, it is preferred to configure the conductor as yarn, twined yarn, twisted yarn, cord or string. The carbon fibers can also be provided as spin-around roving, as braided string or as fibers that are entirely or only in sections adhesively connected with one another. The conductor formed in this manner is flexible and can thus adapt to any bend of the hose without defying thereto.

The yarn can be a multifilament yarn, which consists of a plurality of monofilaments. Then it comprises many carbon fibers that respectively extend continuously over the entire length of the conductor. However, this does not completely exclude that individual carbon fibers have a shorter length.

However, it is also possible to use a staple fiber yarn as yarn, which consists of carbon fibers having a limited length, which is shorter than the entire length of the conductor. Due to the intense lateral contact of the fibers amongst each other, for example due to a spinning process, a good heat transfer in conductor longitudinal direction is achieved anyway.

The conductor can have a constant cross-section over its entire length, wherein the conductor can comprise or consist of carbon fibers from its proximal end up to its distal end. In such a case, the instrument according to the invention can be particularly easily adapted to a desired required length for the specific use. For example, then the instrument is provided in a standard length, which is as large as the maximum required length (for example 3 m). If an instrument of shorter length is required, a section can be separated from the distal end, whereby then the instrument is immediately ready for operation. Also, an instrument with used end, for example thermally damaged or damaged due to tissue deposition, can be immediately made ready for operation again by means of a minimal shortening, that means by cutting of the damaged end.

In both above-mentioned cases, the conductor comprising carbon fibers or consisting of such fibers does not only serve for current conduction in longitudinal direction of the instrument. Rather its distal end serves as electrode for supplying the plasma. Because argon flows around the conductor and thus oxygen is kept away therefrom, the carbon fibers show a long lifetime and no disturbing erosion. Also, no centering of the conductor inside the lumen is required, so that the heat stress of the hose at its distal end in the area of the plasma remains low.

It is to be noted that it is possible to attach a metal electrode on the distal end of the conductor consisting of carbon fibers or comprising such fibers. For example, it can be an uncovered stainless steel electrode, a silver-coated stainless steel electrode, a graphite electrode, an electrode consisting of a material having good heat-conducting characteristics, such as tungsten carbide (hard metal) or an electrode consisting of tungsten or the like. The electrode can be configured in the type of a brush, a pen having a pointed or blunt end, a needle, a loop, or another expedient form.

Independent from the material characteristics and the form of the distal end of the conductor, the latter can have a proximal section, which is formed by a metallic conductor, for example an aluminum wire, a steel wire, or a wire from another metal. The configuration of the wire from aluminum or steel has the advantage that during the combustion of a used instrument, no toxic residual materials are created. Iron, iron oxide, aluminum and aluminum oxide are considered as harmless residual materials.

A conductor is preferably provided with an insulation forming an inner insulation for the instrument, while the hose is an outer insulation. Preferably, the insulation extends over the entire length of the conductor and in this manner guarantees the dielectric strength. The insulation thereby consists preferably from a plastic from the group of polyimides. Also other plastics with high insulation capability and low electrical loss angle can be used.

If the conductor is wound with a plastic band of a suitable material, for example a polyimide, for electrical insulation, the flexibility of the conductor is not remarkably affected, so that the instrument remains very flexible. Therefore, it can also be used in endoscopes if they require a large bend of the instrument.

The winding is preferably a cross-winding, that means while one winding winds in form of a right-hand helix around the conductor, the layer arranged on top or below thereof is wound as left-hand helix. It might well be that the flexibility of the conductor is thereby slightly reduced, however this supports the electrical insulation. Though, also in this case, the flexibility of the conductor is still sufficient in order to allow a bending of the instrument inside an endoscope.

The conductor is preferably radially moveably supported inside the lumen and namely again preferably along its entire length including its distal end. It has shown that a precise centering of the conductor inside the lumen can be omitted. But due to the omitted centering, also a heat-conducting contact between the conductor and the hose is omitted, which in turn serves for thermal relief of the hose. The latter can thus consist of an environmental-friendly plastic, for example a plastic from the group of polylactides or if applicable also from the group of silicone plastics.

The elimination of a centering device, particularly at the distal end of the instrument, also means that the instrument can be shortened as necessary, i.e. simply cut. The cutting face of the cut conductor then forms the electrode.

The hose can also consist of a plastic from the group of polylactides or silicones. Preferably the polymers for production of the hose are obtained from renewable raw materials having a neutral CO₂ footprint.

Further details of advantageous embodiments of the invention are derived from the dependent claims, the drawing and the associated description.

### BRIEF DESCRIPTION OF THE DRAWINGS

The drawing shows:

FIG. 1 an instrument according to the invention connected to a supplying apparatus in a schematic overview illustration,

FIG. 2 a longitudinal cut through the distal end of the instrument according to FIG. 1 not drawn to scale,

FIG. 3 a front view of the instrument according to FIG. 1 not drawn to scale,

FIGS. 4 and 5 modified embodiments of the instrument according to the invention in a longitudinally cut illustration not drawn to scale.

### DETAILED DESCRIPTION

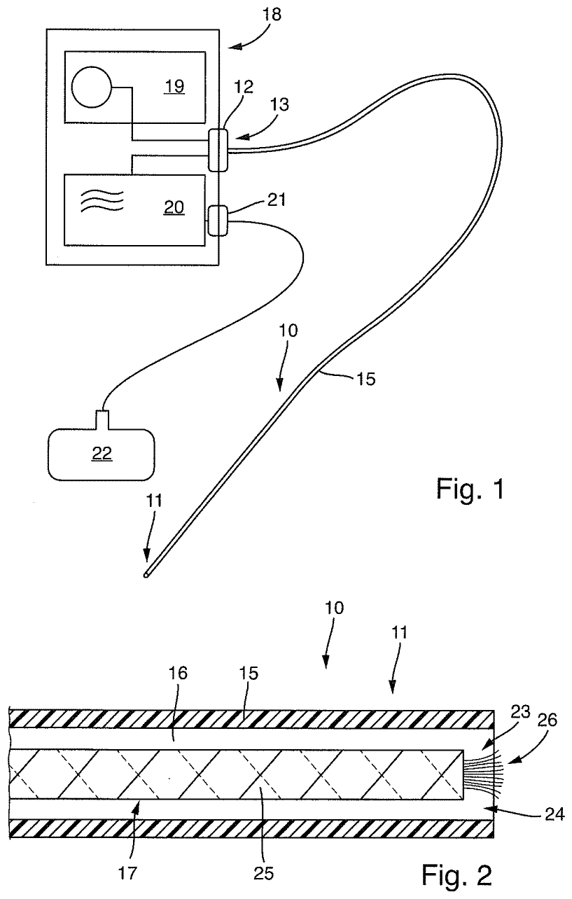

An instrument 10 configured as a flexible probe is illustrated in FIG. 1, having a distal end 11 that can be inserted into a patient through the working channel of a not illustrated endoscope and in this manner can be moved to an operation site. In addition, it comprises a proximal end 13 provided with a connection means, for example a connector 12. However, different to the illustration, instrument 10 can also be configured as a laparoscopic instrument or as an instrument for the open surgical use. If the instrument is configured as flexible probe as illustrated in FIG. 1, it comprises a hose 15 extending from the proximal end 13 up to the distal end 11. If the instrument is a laparoscopic instrument, the hose can be configured as tube shank, i.e. as rigid tube, particularly in the distal section. This applies similarly for an instrument provided for the open surgical use.

Preferably, the hose 15 consists of a plastic from the group of polylactides or from the group of silicones. A lumen 16 extends through the hose 15 from the proximal end 13 up to the distal end 11, in which an electrical conductor 17 is arranged. The conductor 17 extends through the entire length of lumen 16.

The connector 12 connected to the proximal end 13 serves for connection with a supplying apparatus 18, containing a source 19 of gas for supply of lumen 16. The source 19 can comprise, for example, an argon storage as well as pressure control armatures and valves in order to supply lumen 16 in a controlled manner with a desired volume flow rate of the respective gas, for example argon.

In addition, the apparatus 18 comprises an electrical generator 20 having an output pole, which is connected with the conductor 17 by means of connector 12. Another output pole of generator 20 is connected by means of another connector 21 and an electrical line with a neutral electrode 22, which is to be extensively connected to a patient to be treated.

The generator 20 is configured to create a high frequency high voltage suitable for the plasma generation, which can have an amount of multiple hundred volts peak-to-peak. The frequency of the high voltage is preferably between 100 kHz and 5 MHz and is typically approximately 350 kHz. Other frequencies are possible within this range. The voltage can be provided without pulses (CW) without modulation. The generator can also be configured to supply the high voltage in modulated form, for example on-off-switched, that means modulated with a square wave having a constant or varying pulse/pause-ratio. The modulation frequency can be below 100 kHz.

In the present embodiment the electrical conductor 17 is a thread consisting of carbon fibers 23. The carbon fibers 23 can be twisted, twined, braided or otherwise connected and kept together. Thereby, the carbon fibers 23 can be configured as monofilaments. Then they extend respectively over the entire length of the instrument 10 from the connector 12 up to the distal end 11 and the exit opening 24 provided there. The carbon fibers 23 can, however, also be shorter than the entire length of hose 15, whereby the thread formed therefrom is then a staple fiber yarn. The individual fibers can be spun with each other in order to form a thread.

In a preferred embodiment, the electrical conductor formed from the carbon fibers 23 is electrically insulated. The insulation 25 provided for this purpose can consist of a plastic having high electrical insulation capabilities and low electrical loss angle. For example, the insulation can be selected from a plastic from the group of polyimides.

Independent from the material selection, the insulation 25 can have the form of strip-shaped material and can be wound around conductor 17 consisting of parallel arranged carbon fibers 23, of twined fibers, of twisted fibers, of braided or otherwise connected fibers. The insulation 25 can be wound following a helical line around the conductor 17. In a preferred embodiment, the winding of insulation 25 follows a right-hand helix, wherein a second layer following a left-hand helix can be arranged on top or below thereof. The double winding provides an insulation 25 with slightly increased stiffness, which however does not remarkably increase the total stiffness of instrument 10. The winding forming a bandage of conductor 17 consisting of carbon fibers 23, particularly the bandage being wound in double manner and countersense, provides in any case a reliable electrical insulation of conductor 17. Consequently, hose 15 can be configured with a thin wall, because the main insulation of the electrical conductor 17 is already provided by the insulation 25.

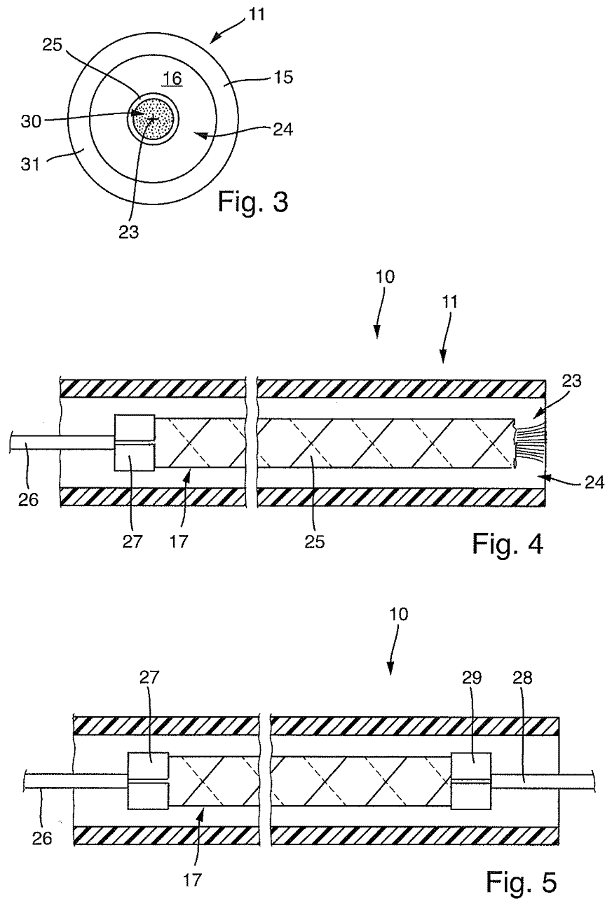

As apparent from FIG. 2 and also FIG. 3, the carbon fibers 23 are uncovered at the distal end of conductor 17, for example in form of a cut surface 30. The cut surface 30 and a respective end or cut surface 31 of hose 15 can be arranged in a common plane or can also be arranged axially offset relative to one another. They can be recreated any time by shortening instrument 10.

At their ends the carbon fibers 23 form the electrode 26 necessary for plasma creation. As it is additionally apparent, lumen 16 is completely free. No centering element exists that would support conductor 17 in the center of the lumen. No element connected with conductor 17 abuts with any pretension against the surface of the hose 15 surrounding lumen 16. At most, the ends of the carbon fibers 23 projecting from the end of conductor 17 can diverge in the type of a brush and can have contact to the wall. Due to the provided electrical resistance, each carbon fiber conveys, however, only proportionally a fraction of the total current depending on the electrical resistance of the carbon fiber, so that the few individual fibers having wall contact only provide a low heat contribution.

The instrument described so far operates as follows:

For start-up, the conductor 12 is connected with apparatus 18 and the instrument 10 is inserted into a patient directly or via a working channel of an endoscope. For treatment the source 19 is activated, whereby an argon flow flows through channel 16 in distal direction. At the same time or after a short period, generator 20 is activated, so that now high voltage is applied to the carbon fibers 23. A spark discharge is formed through the argon stream and thus a plasma stream is formed toward the tissue to be treated. The current thereby flows from the generator via conductor 17 through the plasma and the body of the patient as well as via the neutral electrode 22 back to the generator 20.

If the instrument 10 is thermally damaged in spite of its robustness in the area of the exit opening 24, it can be reset again into a condition ready for use in that the instrument 10 is shortened at its distal end 11 about a few millimeters or centimeters. Thereby the hose 15 and the conductor 17 arranged therein are cut commonly in order to create new cut surfaces 30, 31. As soon as the instrument 10 is then operated again, the insulation 25 melts slightly backward, thereby uncovers the carbon fibers 23 and the instrument 10 is further on operable in unlimited manner.

The same procedure can be carried out if the instrument 10 supplied in standard length is too long and must be shortened, for example about one or multiple decimeters. It can then be cut down to the desired length in that the excess length is separated from the distal end 11.

The used instrument 10 can be combusted, as it is usual for hospital waste if it shall not or cannot be recycled. In the described embodiment it does not contain metallic elements, potentially apart from the area of connector 12, and consists finally only from the elements carbon and hydrogen as well as potentially additionally nitrogen and oxygen. Thus, it can be combusted to non-hazardous residual materials. No toxic waste is created and all the more no persistently durable toxins.

The instrument basically described so far can also be realized in modified form, as for example depicted in FIG. 4. The instrument 10 illustrated in FIG. 4 comprises a conductor 17 that corresponds over a remarkable section of its length, however particularly in the area of the exit opening 24, to the instrument 10 according to FIG. 2 described above. Outside its distal section 11, conductor 17 can also be formed by a metallic conductor 26 though, for example by an aluminum wire, a steel wire, a stainless steel wire or the like. The length of the section of the conductor 17 containing the carbon fibers 23 is preferably at least as long as the section that has to be maximally cut from the distal end of the instrument 10 for shortening to a desired minimum length or for re-establishment of the function.

A crimp connection having a crimp ring 27 or also another suitable connection means serves for connection with the conductor comprising the carbon fibers 23. The crimp ring 27 and the electrical conductor 17 are in turn preferably provided with an electrical insulation that is not further illustrated. Also, this electrical insulation can be configured similar to insulation 25 and can particularly consist of a polyimide. For example, it can be a polyimide hose, a polyimide coating or also a winding with one or more polyimide bands according to the model of insulation 25.

FIG. 5 illustrates another modified embodiment of the instrument 10 according to the invention. It can have a conductor 17 according to the model of embodiment according to FIG. 2, which extends over the entire length of instrument 10 and in total consists of carbon fibers 23. The conductor 17 can, however, also be configured according to the model of the embodiment according to FIG. 4 and can comprise the metallic conductor 26 in its proximal section; both is possible. The particularity of the embodiment according to FIG. 5 consists in that the distal end of the conductor 17 supports a compact electrode 28. This compact electrode 28 can be a needle, a pin or another body of a metal, of an electrically conductive ceramic or of carbon, for example graphite. The compact electrode 28 can in turn be connected by means of a crimp ring 29 or another suitable fixation electrically and mechanically with conductor 17.

For all of the embodiments described above applies that instead of each crimp ring 27, 29 also other suitable attachment means, for example plastic shrink elements or tight windings of wire or a suitable binding material, for example a plastic thread, ceramic fiber, glass fiber or carbon fiber can be used. The metallic conductor 26 as well as the compact electrode 28 can respectively extend over a part of their length into the conductor 17 consisting of carbon fibers and thereby have a rigid mechanical and electrical connection thereto.

An improved instrument 10 for argon plasma surgery comprises a plastic hose 15 and an electrical conductor 17 extending therethrough, which is formed by a thread of carbon fibers 23 or a flexible or rigid body comprising carbon fibers 23. The carbon fibers 23 particularly serve for current conduction and the thermal cooling of the plasma foot point which forms directly at the carbon fibers 23 at the end of the electrical conductor 17 or in a compact body 28 in contact with the latter. The electrical conductor 17 is arranged inside the lumen 16 of hose 15 without being centered and thus without forced contact to the inner wall of hose 15. In doing so, the high thermal conductivity of the conductor 17 in longitudinal direction results in a low thermal stress of hose 15 and thus a long lifetime of the instrument 10. In addition, due to this construction, the instrument 10 can be provided with halogen-free plastics, whereby a thermal disposal after use of the instrument 10 does not cause problems. This applies particularly if the instrument 10 is entirely or largely configured in a metal-free manner.

### REFERENCE SIGNS

10 instrument
11 distal end of instrument 10
12 connector
13 proximal end of instrument 10
15 hose
16 lumen
17 conductor
18 apparatus
19 source
20 generator
21 connector
22 neutral electrode
23 carbon fibers
24 exit opening
25 insulation
26 metallic conductor
27 crimp ring
28 compact electrode
29 crimp ring
30, 31 cut surfaces

1. An instrument (10) for medical plasma treatment of biological tissue, the instrument (10) comprising:
a hose (15) comprising a proximal end (13) and a distal end (11) and at least one lumen (16), wherein the hose is configured to be connected at the proximal end (13) to a gas supply source (19), wherein the hose (15) comprises an exit opening (24) at the distal end (11) thereof;
an electrical conductor (17) that extends through a length of the at least one lumen (16) and ends at or adjacent to the exit opening (24); and
wherein the electrical conductor (17) comprises carbon fibers (23).

2. The instrument according to claim 1, wherein the carbon fibers (23) are predominantly arranged in a longitudinal direction of the at least one lumen (16).

3. The instrument according to claim 1, wherein the electrical conductor (17) is a yarn, a twined yarn, a twine, a cord, or a string.

4. The instrument according to claim 3, wherein the yarn is a multifilament yarn.

5. The instrument according to claim 3, wherein the electrical conductor (17) is a staple fiber yarn.

6. The instrument according to claim 1, wherein the electrical conductor (17) comprises a constant cross-section over its entire length.

7. The instrument according to claim 1, wherein the electrical conductor (17) comprises a cut surface (30) at the exit opening (24).

8. The instrument according to claim 1, wherein the electrical conductor (17) is provided with an insulation (25).

9. The instrument according to claim 8, wherein the insulation (25) is formed to extend over an entire length of the electrical conductor (17).

10. The instrument according to claim 8, wherein the insulation (25) consists of a plastic selected from the group of polyimides.

11. The instrument according to claim 8, wherein the insulation (25) comprises an insulating plastic band that is wound around the electrical conductor (17).

12. The instrument according to claim 1, wherein the electrical conductor (17) is radially movably supported inside the at least one lumen (16).

13. The instrument according to claim 1, wherein the electrical conductor (17) is radially freely movably supported in the distal end (11) of the hose.

14. The instrument according to claim 1, wherein the hose (15) consists of halogen-free plastic material.

15. The instrument according to claim 1, wherein the hose (15) consists of a plastic selected from the group of polylactides or the group of silicones.
